(19) United States
(12) Patent Application Publication
(10) Pub. No.: US 2010/0137651 A1
(43) Pub. Date: Jun. 3, 2010

(54) METHOD FOR PRODUCTION OF A LIQUID COMPOSITION OF AN ORTHO-DIHYDROXYBENZYL COMPOUND OF HIGH PURITY AND SAID COMPOSITION

(75) Inventor: Claude MATHIEU, Corbas (FR)

Correspondence Address: BUCHANAN, INGERSOLL & ROONEY PC, POST OFFICE BOX 1404, ALEXANDRIA, VA, 22313-1404, US

(73) Assignee: Rhodia Chimie, Boulogne Billancourt Cedex (FR)

(21) Appl. No.: 12/698,439

(22) Filed: Feb. 2, 2010

Related U.S. Application Data
(63) Continuation of application No. 10/504,644, filed on Jan. 7, 2005.

(30) Foreign Application Priority Data
Feb. 15, 2002 (FR) 02/01925

(51) Int. Cl. C07C 37/74

(52) U.S. Cl. 568/753

(57) ABSTRACT

The invention relates to a process for the preparation of a liquid composition of an ortho-dihydroxybenzyl compound of high purity characterized in that it consists inpurifying the initial ortho-dihydroxybenzyl compound by distillation and recovering the condensed ortho-dihydroxybenzyl compound in an apparatus resistant to corrosion by the latter, and then mixing it with an organic solvent in a storage tank which is lined or made of a material which does not lead to metallic pollution. The invention also relates to a liquid composition of an ortho-dihydroxybenzyl compound of high purity comprising said compound and an organic solvent of said compound obtained according to said process.

The subject of the present invention is a process for the preparation of a liquid composition of an ortho-dihydroxybenzyl compound of high purity and the composition thus obtained.

The invention relates more particularly to the preparation liquid composition of pyrocatechol of high purity.

Some fields of application, for example electronics, require products of very high purity in particular metallic elements. Thus pyrocatechol or 1,2-dihydroxybenzene is introduced into formulations intended to remove the “photoresists” polymers which are used for the etching of integrated and printed circuits. It is important to not pollute the semi-conductor with metallic elements which would risk creating disturbances.

Pyrocatechol, because of its complexing properties, is also used as chelating agents and as corrosion inhibitors.

Thus there is strong demand in the market for extremely pure products.

It is not easy to obtain such products on an industrial scale. In fact, because of their chelating properties, obtaining such products which are free from impurities is very difficult.

Moreover, pyrocatechol is a product which is commercially available in powder or flake form and which is known to be an irritant and all users must take precautions in terms of industrial hygiene to handle it. As regards a product in the form of a solid, users must undertake handling operations, solution treatments in industries which do not always have equipment which is well suited.

A purer product is generally obtained as a result of distillation and forming operations (crystallization, grinding, flaking etc.) so as to obtain pyrocatechol in a solid form which can be handled. However when it leaves the distillation column and comes into contact with materials found in forming devices it very quickly becomes polluted because of the abrasive properties of pyrocatechol.

Thus the following chemical elements can be found: Al, Sb, As, Ba, Be, Bi, B, Cd, Cl, Ca, Cr, Co, Cu, Ga, Au, Fe, Li, Mg, Mn, Ni, K, Pb, Ag, Na, Sr, Ta, Tl, Sn, Ti, V, Zn, Zr, Hg, l.

Some metallic elements in significant quantities are: iron, aluminium, chromium, magnesium, nickel, calcium, manganese, sodium and potassium.

To overcome this drawback, it is proposed according to EP-A 1 055 657 to produce a dihydroxybenzyl compound with a total metal content of less than 200 ppb which consists in evaporating said compound in a distillation column, condensing the vapour fraction of said compound in a condenser connected to an outlet and recovering the condensed fraction in a storage tank via a conduit connected to the condenser, said condenser has an internal surface constituted by a metallic material comprising at least 25% by weight of nickel and the condensed fraction comes into contact with said surface.

It should be noted that the examples relating to pyrocatechol show a difference in purity of the product obtained of 15 ppb with respect to the use of a standard device which leads to obtaining a product with a total content of metallic impurities of 592 ppb. However, the text does not mention the recovery of the pyrocatechol at the outlet of the condenser.

The subject of the present invention is not to provide pyrocatechol in the form of a solid but in the form of a liquid composition of pyrocatechol of high purity which is easily used by users, without the risk of subsequent pollution.

The invention aims to provide an ortho-dihydroxybenzyl compound of high purity in the form of a liquid at ambient temperature.

By “ambient temperature” is generally meant a temperature in a temperature range from 0° C. to 35° C., preferably comprised between 15° C. and 25° C.

By “high purity” is meant a chemical purity of the initial ortho-dihydroxybenzyl compound determined by gas chromatography of at least 99% and a content of each metallic element of at most 100 ppb, preferably less than 60 ppb.

In the present text, the metallic element content is defined with respect to the ortho-dihydroxybenzyl compound and not with respect to the solution.

By “liquid” according to the invention, viscous products are included but still in the liquid state.

One of the subjects of the invention is constituted by the liquid compositions of an ortho-dihydroxybenzyl compound of high purity.

More specifically, the invention consists of a liquid composition of an ortho-dihydroxybenzyl compound of high purity characterized in that it comprises at least one ortho-dihydroxybenzyl compound of high purity and at least one organic solvent solubilizing said compound used in a sufficient quantity to obtain a liquid composition.

Another subject of the invention is the process for the preparation of said liquid composition characterized in that it consists in:
purifying the initial ortho-dihydroxybenzyl compound by distillation and recovering the condensed ortho-dihydroxybenzyl compound in an apparatus resistant to the corrosion of the latter,
then mixing it with an organic solvent in a storage tank which is lined or made of a material which does not lead to metallic pollution.

The present invention aims to provide liquid compositions of an ortho-dihydroxybenzyl compound of high purity comprising at least one ortho-dihydroxybenzyl compound and at least one organic solvent.

Preferably, the compositions of the invention comprise at least one or more ortho-dihydroxybenzyl compounds of formula (I):

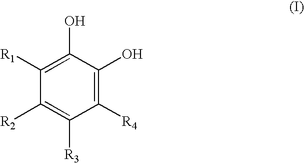

in said formula:
R₁, R₂, R₃ and R₄, identical or different, represent a hydrogen atom, an alkyl group, an alkoxy group, a halogen atom, a trifluoromethyl group, a nitro group or a carboxylic COOH group, a CHO group,
at least one substituent from R₁, R₂, R₃ and R₄ represents a hydrogen atom,
at most two substituents from R₁, R₂, R₃ and R₄ represent a halogen atom, a trifluoromethyl group, a nitro group or a carboxylic COOH group, a CHO group.

By “alkyl” is meant according to the invention a linear or branched or cyclic hydrocarbon chain with 1 to 12 carbon atoms, such as for example, methyl, ethyl, n-propyl, isopropyl, n-butyl, sec-butyl, tert-butyl and octyl. The C₁-C₄ alkyl groups are preferred.

By “alkoxy” is meant according to the invention an alkyl-oxy group where the alkyl group is as defined previously. Preferably, the alkoxy group is an isopropoxy group.

By “halogen” is meant the fluorine, chlorine or bromine atom.

A group of preferred ortho-dihydroxybenzyl compounds is constituted by compounds of formula (I) in which at least three substituents from R₁, R₂, R₃ and R₄ represent a hydrogen atom. Among these, those for which the fourth substituent R₁, R₂, R₃ or R₄ is chosen from a hydrogen atom, a C₁-C₁₂ alkyl group, preferably C₁-C₄ and a C₁-C₁₂ alkoxy group, preferably C₁-C₄, are even more advantageous.

A group of preferred compounds is that constituted by pyrocatechol, 3-methylpyrocatechol, 4-methylpyrocatechol, 3-isopropylpyrocatechol, 3-butyl-5-methylpyrocatechol, 4-tert-butylpyrocatechol, 2-chloropyrocatechol, 4-chloropyrocatechol, 3,5 -di-tert-butylpyrocatechol, 4,6-di-tert-butylpyrocatechol, 3-octyl-5-methylpyrocatechol, 4-isopropoxypyrocatechol, 3,6-diisopropylpyrocatechol, 4-nitropyrocatechol, dihydroxy-3,4 benzoic aldehyde.

Among the above compounds, pyrocatechol is the preferred compound.

It is noted that all the different ortho-dihydroxybenzyl compounds are commercially available or easily prepared by a person skilled in the art.

As regards the organic solvent, he makes his choice taking into account several requirements.

This solvent should be inert vis-à-vis the ortho-dihydroxybenzyl compound and capable of solubilizing it.

It is also chosen with a view to the use envisaged.

A mixture of organic solvents can be used.

Preferably a polar organic solvent is used.

There may be mentioned in particular as specific examples:
the optionally hydroxylated nitrogenous type compounds such as: ethylene diamine, NMP (N-methylpyrrolidone), pyridine, MEA (monoethanolamine), diethanolamine, triethanolamine, tert-butyl diethanolamine, isopropanolamine, 2-amino-1-propanolamine, 3-amino-1-propanolamine, isobutanolamine, 2-amino-2-ethoxyethanol, DGA [diglycolamine or 2-(2-aminoethoxy)ethanol],
alcohol and/or ether and/or ester type compounds such as: ethylene glycol, propylene glycol, TEG [triethylene glycol], glyme, diglyme, PGMEA [propylene glycol monomethyl ether acetate or 2-(1-methoxy)propyl acetate], PGME [propylene glycol monomethylether], ethyl lactate, anisole, methyl adipate, cyclopentanol,
hydrocarbon type compounds such as toluene, xylene, mesitylene,
ketone type compounds such as methyl ethyl ketone, 2-pentanone, cyclopentanone, cyclohexanone, mesityl oxide,
dimethyl sulphoxide.

In order to obtain a liquid composition the following are preferably used:
from 5 to 80% by weight of an ortho-dihydroxybenzyl compound,
from 20 to 95% by weight of an organic solvent.

The preferred compositions of the invention comprise:
from 5 to 50% by weight of an ortho-dihydroxybenzyl compound,
from 50 to 95% by weight of an organic solvent.

The invention applies very well to the preparation of liquid compositions of pyrocatechol of high purity.

Preferably they comprise from 5 to 50%, preferably. 15 to 40% of pyrocatechol and from 50 to 95%, preferably from 60 to 85% of an organic solvent.

The preferred compositions according to the invention comprise from 5 to 50% by weight of pyrocatechol and from 50 to 95% by weight of DGA or MEA.

The liquid compositions of pyrocatechol obtained have the purity defined above.

Advantageously, the total of all the metallic elements is less than 100 ppb, preferably less than 80 ppb.

According to the invention, a liquid composition of an ortho-dihydroxybenzyl compound with high purity as regards metallic elements is obtained by implementing the process according to the invention.

More specifically, the process starts with an ortho-dihydroxybenzyl compound which is generally commercially available of high purity, usually higher than 50%.

It is purified by distillation.

It is noted that said compound can be in a mixture with other compounds. This is the case for hydroquinone and pyrocatechol which are produced simultaneously by hydroxylation of phenol with hydrogen peroxide, in the presence of a strong acid generally perchloric acid or sulphuric acid.

Distillation takes place at a temperature comprised between 120° C. and 200° C. under a pressure ranging from 30 to 100 mm of mercury.

It is carried out in a standard retort.

A person skilled in the art is perfectly capable of choosing the means to be used according to the initial ortho-dihydroxybenzyl compound.

The following should be noted: the size (in particular the diameter) of the distillation columns depends on the circulating flow and the internal pressure. Their size will thus principally be determined according to the flow rate of mixture to be treated. The internal parameter of the number of theoretical stages is determined in particular by the purity of the initial compound and the purity of the product which is to be obtained at the distilling head.

It is specified that the columns can be packed either with trays or regular packing, as a person skilled in the art knows perfectly well.

The installation having been determined, a person skilled in the art adjusts the operating parameters of the column.

Thus the distillation column can advantageously, but not limitatively, be a column with the following specifications:
number of theoretical stages: from 1 to 50, preferably from 20 to 30,
reflux ratio R comprised between 0.5 and 10, preferably between 3 and 6.

At the bottom of the column, the undesirable products are recovered, in particular the organic products with higher boiling point containing all of the metallic traces.

At the head of the column, a gaseous phase constituted by the purified ortho-dihydroxybenzyl compound is recovered.

The gaseous phase is cooled and is transformed into liquid form by cooling to a temperature slightly higher than its crystallization point, for example higher than 5 to 20° C., preferably higher than 10° C.

This operation is carried out by passage in a condenser which is a standard apparatus for example a tubular exchanger fed with water or with a fluid (generally an oil) maintained at a temperature close to the chosen cooling temperature.

According to one characteristic of the process of the invention, it is important that the head of the distillation column and the condenser do not lead to metallic pollution.

To this end, stainless steels are advantageously chosen, such as austenitic steels and more particularly the stainless steels 304, 304 L, 316 or 316L.

A steel is used which has at most 22% by weight of nickel, usually between 6 and 20%, preferably between 8 and 14%.

The steels 304 and 304 I have a nickel content varying between 8 and 12% and the steels 316 and 316 L have a nickel content varying between 10 and 14%.

Such steels are commonly used in industry.

For the definition of austenitic steels, reference can be made to the work of Robert H. Perry et al, [Perry's Chemical Engineers' Handbook, Sixth Edition (1984), page 23-11].

The ortho-dihydroxybenzyl compound is recovered closest to its distillation point and it is mixed as quickly as possible with an organic solvent free from metallic elements.

To this end, the purified product is conveyed by pipes made of stainless steel such as described previously or made of plastic then mixing takes place with the organic solvent in a storage tank made of stainless steel or plastic.

One or more storage tanks can be envisaged. In this case, the product can be conveyed by pumps made of stainless steel.

It is noted that, as the residence time of the ortho-dihydroxybenzyl compound is very short in these tubes, the materials should be resistant to corrosion but no mechanical demand is made of them.

When the material used is stainless steel, it is necessary that the tool concerned is rinsed beforehand, once or several (3 or 4) times, with the ortho-dihydroxybenzyl compound which is desired to be obtained in solution.

It is also necessary to note that any prolonged storage advantageously takes place in plastic containers.

As plastic materials, a polymeric compound is chosen which is resistant to corrosion by the composition of the invention. In particular materials such as PTFE (polytetrafluoroethylene or Teflon) or PFA (perfluoroalkyl resins), high-density polyethylene can be mentioned. Using an equivalent material is not beyond the scope of the invention.

Preferably, the purified ortho-dihydroxybenzyl compound is advantageously conveyed by a pipe made of plastic or comprising a plastic lining into a plastic storage tank in which mixing with the organic solvent is carried out.

Thus the liquid composition of the ortho-dihydroxybenzyl compound of high purity is recovered directly.

In relation to the other solutions advocated in the literature, the presentation in liquid form has the advantage of eliminating the part where the solid is formed which requires expensive materials with high nickel content and packing and unpacking equipment for solids.

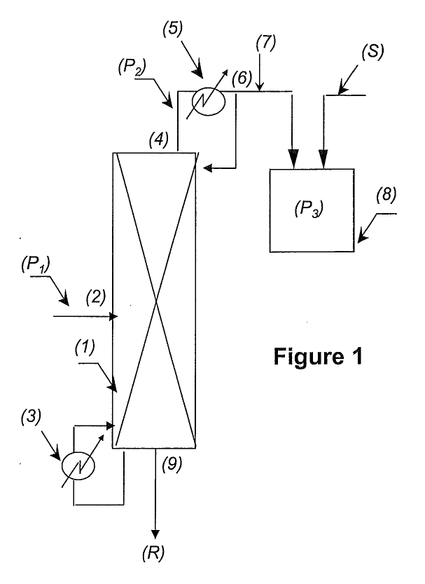

In attached FIG. 1 a preferred embodiment of the invention is shown.

The installation allowing the preparation of compositions of the invention comprises a first column (1) equipped with means for admitting (2) the ortho-dihydroxybenzyl compound to be purified, in liquid form, (P₁) preferably sent by a pump into the column, generally into the middle of the column.

The column is equipped in its lower part with heating means [boiler] (3) and in its upper part with means for evacuation (4) of the gaseous phase (P₂) which passes through a condenser (5) and at its outlet (6), a fraction determined by the reflux ratio is sent back into the column and the other fraction is sent via a conduit made of stainless steel, or preferably made of plastic (7) into a storage tank (8) made of stainless steel or plastic into which the organic solvent (S) is added by gravity, preferably without passing through a pump.

The solvent (S) can be loaded beforehand into (8) before the addition of the ortho-dihydroxybenzyl compound.

Thus there is direct recovery of the liquid composition of an ortho-dihydroxybenzyl compound of high purity in the tank (8) and of the undesirable products at the bottom of the column (9).

Some embodiment examples of the invention and comparative examples are given below.

### EXAMPLE 1

Reference is made to FIG. (1) to describe the example.

In a packed distillation column (1), with a diameter of approximately 1.0 metre and a height of approximately 6 metres made of stainless steel 304 L, a mixture of hydroquinone and pyrocatechol comprising the initial pyrocatechol at a degree of purity of approximately 45% by weight and containing more than 100 ppb of iron is introduced at (2) at a flow rate of 500 kg/h.

Heating is carried out using a boiler (3) situated at the foot of the column to a temperature in the order of 210° C.

The reflux ratio of the column is adjusted to obtain the desired purity ratio.

Vapour is fed to the boiler (3) at a flow rate which is adjusted as a function of the reflux ratio.

The ratio is chosen to be of the order of 3.

At the foot of the column (9), the undesired products, namely hydroquinone and the metallic elements, are recovered.

The gaseous phase recovered at the head of the column (4) is sent to a condenser (5). It is condensed at a temperature of 120° C.

As it leaves the condenser (6) part of the condensed flow is sent back into the column according to the reflux ratio and the other part is sent to a container made of PFA (8) by a pipe made of PFA (7).

The pyrocatechol leaving the condenser and entering the container containing the solvent has a purity higher than 99.5% by weight and contains the following elements: Sb; As; Ba; Be; Bi; B; Cd; Cr; Co; Cu; Ga; Au; Li; Mn; Ni; Pb; Ag; Sr; Ta; Tl; Sn; V; Zn; Zr; Hg; I; each of the elements being at less than 0.1 ppb

Al: 5 ppb

Ca: 7.4 ppb

Fe: 16 ppb

Mg: 2.8 ppb

K: 7.9 ppb

Na: 35 ppb

Ti: 0.3 ppb

or a total of less than 80 ppb for the 33 compounds analyzed.

The analysis was done with one of the most effective ICP/MS (Induction Couple Plasma/Mass Spectrometry) methods.

The solvent DGA is introduced into, the container (8) beforehand at the rate of 800 kg, pyrocatechol then being recovered until a volume of 200 kg is obtained which corresponds to obtaining of a solution of pyrocatechol at 20% by weight.

### EXAMPLE 2

After 1000 hours of operating according to Example 1, a pyrocatechol with the following purity is obtained: the following elements each at less than 0.1 ppb: I; Sb; As; Ba; Be; Bi; B; Cd; Co; Cu; Ga; Au; Li; Mn; Ni; Pb; Ag; Sr; Ta; TI; Sn; Ti; V; Zn; Zr; Hg;

K: 12 ppb

Mg: 2 ppb

Ca: 12 ppb

Fe: 6 ppb

Na: 26 ppb

Cr: 24 ppb

The total of the 33 elements is less than 85 ppb.

### COMPARATIVE EXAMPLE 3

Starting with a purified pyrocatechol. The content of each metal is less than 50 ppb.

The distilled pyrocatechol is subjected to flaking on a drum flaker, the knife being made of bronze, the drum of stainless steel.

The flaked pyrocatechol obtained has an iron content greater than 100 ppb.

### EXAMPLE 4

A solution of pyrocatechol at 21.5% by weight in monoethanolamine MEA is prepared.

To this end, Example 1 is repeated but monoethanolamine is used.

The pyrocatechol at the outlet of the condenser has a purity higher than 99.5% by weight and contains the following elements:

Al: 2 ppb

Fe: 13 ppb

Cr: 15 ppb

Ca: 5 ppb

Mg: 2 ppb

K: 5 ppb

Na: 8 ppb

Bi: 5 ppb

Pb: 2 ppb

The other elements are at less than 0.1 ppb.

### EXAMPLE 5

Results similar to those of Example 1 are obtained using purified pyrocatechol by distillation on a column with a regular packing of stainless steel (Sulzer BX) with a diameter of 1 meter. The gaseous phase recovered at the head of the column is sent to a condenser with a 75 m² exchange surface. The pyrocatechol leaving the condenser passes through a first storage tank made of stainless steel (304), a pump made of stainless steel, a pipe made of stainless steel (316 L) 200 meters in length, a second storage tank made of stainless steel (316 L), a pump made of stainless steel for transferring it to a storage tank made of plastic (high-density polyethylene) containing the solvent.

It is noted that in order to obtain the high purity pyrocatechol solution, it is necessary to rinse the installation beforehand with pure pyrocatechol.

Before being mixed with DGA solvent the pyrocatechol has a purity higher than 99.5% by weight and contains the following elements: Sb; As; Ba; Be; Bi; B; Cd; Cr; Co; Cu; Ga; Au; Li; Mn; Ni; Pb; Ag; Sr; Ta; TI; Sn; V; Zn; Zr; Hg; I; each of the elements being at less than 0.1 ppb

Al: 4.1 ppb

Ca: 4.3 ppb

Fe: 13 ppb

Mg: 1.5 ppb

K: 3.9 ppb

Na: 69 ppb

Ti: 1 ppb

or a total of less than 100 ppb for the 33 compounds analyzed,

A solution of pyrocatechol at 20% by weight in DGA is obtained.

1. A process for the preparation of a liquid composition of an ortho-dihydroxybenzyl compound of high purity comprising the consecutive steps of:
a) purifying the initial ortho-dihydroxybenzyl compound by distillation,
b) condensing the purified ortho-dihydroxybenzyl compound obtained in step a) in an apparatus resistant to corrosion by the latter,
c) recovering the condensed ortho-dihydroxybenzyl compound obtained in step b) closest to its distillation point,
d) mixing the recovered ortho-dihydroxybenzyl compound in step c) with an organic solvent in a storage tank which is lined or made of a material which does not lead to metallic pollution, and, then
e) obtaining the liquid composition of the ortho-dihydroxybenzyl compound having less than 100 ppb metallic elements.

2. The process according to claim 1, wherein the distillation step a) and the condensation step b) of the purified ortho-dihydroxybenzyl compound take place in an apparatus made of austenitic steel.

3. The process according to claim 2, wherein the steel is stainless steel 304, 304 L, 316, or 316 L.

4. The process according to claim 3, wherein the steel has at most 22% by weight of nickel.

5. The process according to claim 4, wherein the steel has from 8 to 14% by weight of nickel.

6. The process according to claim 1, wherein the purified ortho-dihydroxybenzyl compound obtained in step a) is conveyed by a pipe made of stainless steel or a pipe made of plastic or comprising a plastic lining.

7. The process according to claim 6, wherein the step d) of mixing takes place in a storage tank made of stainless steel or plastic.

8. The process according to claim 1, wherein the steps a) and b) are carried out in an apparatus made of stainless steel, and the purified ortho-dihydroxybenzyl compound is conveyed by a pipe made of stainless steel or by a pipe made of plastic or comprising a lining made of plastic and step d) takes place in a storage tank made of stainless steel or plastic.

9. The process according to claim 8, wherein the apparatus made of stainless steel is subjected to one or more rinses carried out using the ortho-dihydroxybenzyl compound which is sought to be placed in solution.

10. The process according to claim 7, wherein the plastic is polytetrafluoroethylene, a perfluoroalkyl resin, or a high-density polyethylene.

11. The process according to claim 1, wherein the ortho-dihydroxybenzyl compound corresponds to formula (I): 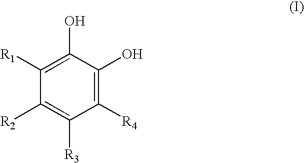
wherein:
R₁, R₂, R₃ and R₄, identical or different, represent a hydrogen atom, an alkyl group, an alkoxy group, a halogen atom, a trifluoromethyl group, a nitro group, a carboxylic COOH group, or a CHO group,
at least one substituent from R₁, R₂, R₃ and R₄ represents a hydrogen atom, and
at most two substituents from R₁, R₂, R₃ and R₄ represent a halogen atom, a trifluoromethyl group, a nitro group, a carboxylic COOH group, or a CHO group.

12. The process according to claim 11, wherein the ortho-dihydroxybenzyl compound corresponds to formula (1), wherein at least three substituents from R₁, R₂, R₃ and R₄ represent a hydrogen atom; the fourth substituent R₁, R₂, R₃ or R₄ being a hydrogen atom, a C₁-C₁₂ alkyl group, or a C₁-C₁₂ alkoxy group.

13. The process according to claim 11, wherein the ortho-dihydroxybenzyl compound is pyrocatechol, 3-methylpyrocatechol, 4-methylpyrocatechol, 3-isopropylpyrocatechol, 3-butyl-5-methylpyrocatechol, 4-tert-butylpyrocatechol, 2-chloropyrocatechol, 4-chloropyrocatechol, 3,5-di-tert-butylpyrocatechol, 4,6-di-tert-butylpyrocatechol, 3-octyl-5-methylpyrocatechol, 4-isopropoxypyrocatechol, 3,6-diisopropylpyrocatechol, 4-nitropyrocatechol, or dihydroxy-3,4 benzoic aldehyde.

14. The process according to claim 1, wherein the organic solvent is ethylene diamine, N-methylpyrrolidone, pyridine, monoethanolamine, diethanolamine, triethanolamine, tert-butyl diethanolamine, isopropanolamine, 2-amino-1-propanolamine, 3-amino-1-propanolamine, isobutanolamine, 2-amino-2-ethoxyethanol, diglycolamine, 2-(2-aminoethoxy)ethanol, ethylene glycol, propylene glycol, triethylene glycol, glyme, diglyme, propylene glycol monomethyl ether acetate, 2-(1-methoxy)propyl acetate, propylene glycol monomethylether, ethyl lactate, anisole, methyl adipate, cyclopentanol, toluene, xylene, mesitylene, methyl ethyl ketone, 2-pentanone, cyclopentanone, cyclohexanone, mesityl oxide, or dimethyl sulphoxide.

15. The process according to claim 1, wherein the liquid composition comprises from 5 to 80% by weight of the ortho-dihydroxybenzyl compound and from 20 to 95% by weight of the organic solvent.

16. The process according to claim 15, wherein the liquid composition comprises from 5 to 50% by weight of high-purity pyrocatechol and from 50 to 95% by weight of 2-(2-aminoethoxy)ethanol or monoethanolamine.

17. The process according to claim 1, wherein the liquid composition comprises less than 80 ppb metallic elements.

18. The process according to claim 1, wherein the liquid composition comprises less than 60 ppb metallic elements.

19. A process for the preparation of a liquid composition of an ortho-dihydroxybenzyl compound of high purity comprising the consecutive steps of:
a) purifying the initial ortho-dihydroxybenzyl compound by distillation,
b) condensing the purified ortho-dihydroxybenzyl compound obtained in step a) in an apparatus resistant to corrosion by the latter,
c) recovering the condensed ortho-dihydroxybenzyl compound obtained in step b) closest to its distillation point,
d) mixing the recovered ortho-dihydroxybenzyl compound in step c) with an organic solvent in a storage tank which is lined or made of a material which does not lead to metallic pollution, and, then
e) obtaining the liquid composition of the ortho-dihydroxybenzyl compound having less than 100 ppb metallic elements,
wherein the distillation step a) and the condensation step b) of the purified ortho-dihydroxybenzyl compound take place in an apparatus made of stainless steel 304, 304 L, 316, or 316 L, and
wherein the steel has at most 22% by weight of nickel.
